(19) United States
(12) Patent Application Publication
(10) Pub. No.: US 2025/0025074 A1
(43) Pub. Date: Jan. 23, 2025

(54) WEARABLE OPTICAL E-TATTOO FOR ARTERIAL AND VENOUS BLOOD OXYGENATION MONITORING

(71) Applicant: BOARD OF REGENTS, THE UNIVERSITY OF TEXAS SYSTEM, Austin, TX (US)

(72) Inventors: Nanshu Lu, Austin, TX (US); Philip Tan, Austin, TX (US); Eric Wang, Austin, TX (US); Sarnab Bhattacharya, Austin, TX (US)

(21) Appl. No.: 18/773,701

(22) Filed: Jul. 16, 2024

Related U.S. Application Data
(60) Provisional application No. 63/527,933, filed on Jul. 20, 2023.

(51) Int. Cl. A61B 5/1455; A61B 5/00; A61B 5/024

(52) U.S. Cl. CPC A61B 5/14552; A61B 5/02427; A61B 5/6833; A61B 2562/043; A61B 2562/164

(57) ABSTRACT

Disclosed and described herein are systems, methods and device for providing simultaneous non-invasive arterial and venous oxygen saturation. An ultra-thin and self-adherable optical electronic tattoo measures blood absorbance changes from multiple locations. Another sensor may be included to provide self-calibration. Using algorithms, the signals from the blood from the artery and vein can be better separated and yield accurate blood oxygen assessments.

### CROSS-REFERENCE TO RELATED APPLICATION

This application claims priority to and benefit of U.S. provisional patent application Ser. No. 63/527,933 filed Jul. 20, 2023, which is fully incorporated by reference and made a part hereof.

### GOVERNMENT SUPPORT

This invention was made with government support under Grant no. W911NF-19-2-0333 awarded by the Army Research Office. The government has certain rights in the invention.

### BACKGROUND

Blood oxygenation saturation (SxO₂) is a patient metric used for detecting various respiratory and metabolic complications. Arterial blood oxygenation (SaO₂) indicates the sufficiency of the respiratory system; specifically, how well oxygen can be transferred from the lungs to the blood. SaO₂ can be easily approximated noninvasively by measuring peripheral blood oxygenation (SpO₂) with a clip-on pulse oximeter. While widely used to assess tissue oxygenation, SpO₂ can only provide an assessment of local hypoxemia (low arterial blood oxygenation). Oxygen extraction is just as important for sustaining normal metabolic activity. Inadequate oxygen uptake by the tissue resulting in higher oxygenation in the venous blood is a symptom of severe sepsis, septic shock, fever and physical exertion. Continuous monitoring can help detect elevated levels of SvO₂, which has been correlated with increased mortality rates in septic shock patients. However, methods for accurately and noninvasively assessing venous oxygen saturation (SvO₂), indicating the oxygen level remaining in the blood post-tissue perfusion, have yet to be developed.

SvO₂ represents the amount of oxygen returning to the heart and therefore gives insight into how much oxygen is consumed by the tissue. Oxygen debt, critical in severely ill and postoperative patients, occurs when tissue oxygen delivered does not meet their metabolic demands. This mismatch can lead to global tissue hypoxia, often undetectable by standard physiological measurements such as arterial and central venous pressures, heart rate, and urine output. Many pathologic conditions, such as infection and sepsis, lead to profound changes in metabolic performance and SvO₂ must be measured to ensure tissue oxygen demand is met by sufficient oxygen delivery. For example, infectious disease tends to increase oxygen consumption due to increases in metabolic rate and fever. This can cause further complications when oxygen delivery is compromised, such as with respiratory or cardiac diseases. Conversely, during sepsis, organ failure induced by severe tissue hypoxia is caused not by poor oxygen delivery but by the inability of cells to efficiently extract oxygen from the blood (e.g., mitochondrial dysfunction). In both cases, SaO₂ may stay at normal levels.

Arterial and venous oxygenations (SaO₂ and SVO₂, respectively) remain an elusive biometric to measure noninvasively. Unfortunately, venous oxygenation can only conventionally be measured accurately with a pulmonary artery or central venous catheter. This process is costly, invasive, and risky. Traditionally, SᵥO₂ is measured using Swan-Ganz catheterization in the pulmonary artery, providing a detailed view of the body's oxygen use and highlighting discrepancies between oxygen delivery and tissue demand. The invasiveness and technical complexity of Swan-Ganz catheterization and central venous lines often limit their use to critically ill patients. Additionally, in trauma situations with low blood pressure, veins tend to collapse, making line placement challenging. Despite being less intrusive, central venous lines still carry risks such as catheter-related infections

The most common attempt at a surrogate for venous blood oxygenation is cerebral Near Infrared Spectroscopy (NIRS). While this method has seen some clinical adoption due to its noninvasive and ease of use, it is error prone as it must assume fixed ratios of arterial and venous blood volumes. Other approaches include observing the low-frequency (<0.5 Hz) components of the photoplethysmography (PPG) signal. This assumes that these originate from mechanical disturbances of the venous system. These assumptions and other inherent issues have prevented both methods from achieving sufficient accuracy for widespread use.

A fundamental assumption of PPG and pulse oximetry is that only the arterial blood induces a pulsatile or alternating current (AC) signal. As a result, by analyzing only the pulsatile absorptivity changes of the tissue, arterial blood oxygenation alone will be isolated. However, this assumption may not hold outside of peripheral vasculature (e.g., fingertip, ear lobe, etc.). Furthermore, PPG probes typically require clips or straps to hold to the skin. This limits them to thin anatomical locations (e.g., finger, wrist). Additionally, the applied pressure from clips or straps tend to collapse the vein due to its compliance and low internal pressure.

Therefore, what is desired are non-invasive systems and methods to use the venous and arterial pulses to simultaneously extract SaO₂ and SvO₂, thus enabling noninvasive oxygen consumption estimation that overcome challenges in the art, some of which are described above.

### SUMMARY

Recent work has suggested that a venous pulse can be measured with a reflectance PPG probe placed over the jugular vein [see I. Garcia-López and E. Rodriguez-Villegas, “Extracting the Jugular Venous Pulse from Anterior Neck Contact Photoplethysmography,” Scientific Reports, vol. 10, no. 1, p. 3466, 2020/02/26 2020, which is fully incorporated by reference]. Other recent work characterizes how the PPG waveform morphology changes when the PPG sensor is shifted laterally over the carotid artery and jugular vein [see P. Tan, S. Tamma, S. Bhattacharya, J. Tunnell, and N. Lu, “Wearable Optical E-Tattoo for Deep Neck Hemodynamic Monitoring,” in 2022 IEEE/ACM Conference on Connected Health: Applications, Systems and Engineering Technologies (CHASE), 17-19 Nov. 2022 2022, pp. 118-122, which is fully incorporated by reference and made a part hereof].

Therefore, disclosed and described herein are device, systems and methods to use the venous and arterial pulses to simultaneously extract SaO₂ and SvO₂. In particular, disclosed and described herein are device, systems and methods for non-invasive extraction of both arterial and venous oxygenation. A set or an array of photoplethysmography (PPG) sensors are laminated directly over the two blood vessels—an artery and a vein. In some instances, another PPG sensor may be worn at a peripheral site (e.g., fingertip, earlobe, toe) to provide calibration. The set of PPG sensors over the vessels provide either (a) a 1D distribution of absorbance changes, or (b) a 2D image of absorbance changes. By utilizing multiple optical wavelengths and comparing the absorbance changes per wavelength for each location, a rough map of oxygenations is created. A proprietary spatial filtering and source separation algorithm is then used to localize the oxygenations and increase oxygenation reading accuracy. To implement this method, a lightweight and skin-adherable electronic tattoo containing an array of PPG sensors that can obtain simultaneous arterial and venous pulse waveforms without applying any pressure to the vessels is provided and described.

Disclosed and described herein is a pressureless, non-invasive system for simultaneous extraction and measurement of arterial and venous blood oxygenation. One embodiment of the system comprises a flexible electronic tattoo. The flexible electronic tattoo comprises a flexible conformable substrate, wherein the substrate is configured to conform to and adhere to a location on a skin of a wearer; a plurality of optical oxygenation sensors mounted on the substrate, wherein the optical oxygenation sensors each non-invasively obtain information related to in-situ blood oxygenation from within a vein and/or artery of the wearer; a communications interface mounted on the interface, wherein the communications interface is in communication with the plurality of optical oxygenation sensors; and a power source mounted on the flexible conformable substrate. The system further comprises one or more processors, wherein the one or more processors receive the information related to the in-situ blood oxygenation from within the vein and/or artery of the wearer through the communications interface and simultaneously extract venous blood oxygenation (SvO₂) and arterial blood oxygenation (SaO₂) from the information related to the in-situ blood oxygenation from within the vein and/or artery of the wearer.

In one aspect of the system, the one or more processors make a determination about oxygen saturation of a localized area proximate the location on the skin of the wearer where the electronic tattoo is adhered based on observed optical absorptions. The localized area may comprise, for example, the neck, chest, back, stomach, upper arm, upper leg, or lower leg of the wearer.

In some instances of the system, the determination about oxygen saturation comprises a one-dimensional (1D) distribution of absorbance changes, or a two-dimensional (2D) image of absorbance changes of the localized area.

In some instances of the system, the information related to the in-situ blood oxygenation from within the vein and/or artery of the wearer from each of the plurality of optical oxygenation sensors is used to modify extracted venous blood oxygenation (SvO₂) and arterial blood oxygenation (SaO₂) estimations from nearby sensors (spatial filtering and source separation). This is required because the light diffuses/scatters through a large volume of tissue and the artery and vein are very close to each other, resulting in crosstalk or a mixture of signals.

In some instances, the system may further comprise a peripheral sensor, separate from the flexible electronic tattoo, wherein the peripheral sensor non-invasively obtains oxygenation information related to a peripheral site of the wearer and provides it to the one or more processors, wherein the one or more processors calibrate one or more of the plurality of oxygenation sensors based on the oxygenation information about the peripheral site. This calibration process involves computing the differential pathlength factor, which is crucial for accurate blood oxygen extraction. The differential pathlength factor can vary from person to person, making this calibration step essential for personalized and precise measurements. As examples, the peripheral site may comprise a fingertip, earlobe or toe of the wearer.

In some instances, the more processors are not mounted on the flexible electronic tattoo and/or the peripheral sensor.

In some instances of the system, each of the plurality of oxygenation sensors and the peripheral sensor comprise multiple photoplethysmography (PPG) sensors.

In some instances of the system, the plurality of oxygenation sensors, and the peripheral sensor communicate wirelessly with the one or more processors.

In some instances of the system, the flexible electronic tattoo does not apply any pressure to the location on the skin of the wearer.

In some instances of the system, the information related to the in-situ blood oxygenation from within the vein of the wearer and/or the information related to the in-situ blood oxygenation from within the artery of the wearer comprise of venous and arterial waveforms respectively.

Another aspect comprises a method for using a pressureless, non-invasive system for simultaneous extraction and measurement of arterial and venous blood oxygenation. One embodiment of the method comprises attaching flexible electronic tattoo to an epidermis of a wearer, proximate an artery and a vein of the wearer. The flexible electronic tattoo comprises a flexible conformable substrate, wherein the substrate is configured to conform to and adhere to a location on a skin of a wearer; a plurality of optical oxygenation sensors mounted on the substrate, wherein the optical oxygenation sensors each non-invasively obtain information related to in-situ blood oxygenation from within a vein and/or artery of the wearer; a communications interface mounted on the interface, wherein the communications interface is in communication with the plurality of optical oxygenation sensors; and a power source mounted on the flexible conformable substrate. The method further comprises receiving, by one or more processors, the information related to the in-situ blood oxygenation from within the vein and/or artery of the wearer through the communications interface; and simultaneously extracting, by the one or more processors, venous blood oxygenation (SvO₂) and arterial blood oxygenation (SaO₂) from the information related to the in-situ blood oxygenation from within the vein and/or artery of the wearer.

In some instances of the method, the one or more processors make a determination about oxygen saturation of a localized area proximate the location on the skin of the wearer where the electronic tattoo is adhered based on observed optical absorptions. The localized area may comprise the neck, chest, back, stomach, upper arm, upper leg, or lower leg of the wearer.

In some instances of the method, the determination about oxygen saturation comprises a one-dimensional (1D) distribution of absorbance changes, or a two-dimensional (2D) image of absorbance changes of the localized area.

In some instances of the method, the information related to the in-situ blood oxygenation from within the vein and/or artery of the wearer from each of the plurality of optical oxygenation sensors is used to modify extracted venous blood oxygenation (SvO₂) and arterial blood oxygenation (SaO₂) from nearby sensors (spatial filtering and source separation). This is required because the light diffuses/scatters through a large volume of tissue and the artery and vein are very close to each other, resulting in crosstalk or a mixture of signals.

In some instances of the method, a peripheral sensor, separate from the flexible electronic tattoo is provided, wherein the peripheral sensor non-invasively obtains oxygenation information related to a peripheral site of the wearer and provides it to the one or more processors, wherein the one or more processors calibrate one or more of the plurality of oxygenation sensors based on the oxygenation information about the peripheral site. As examples, the peripheral site may comprise a fingertip, earlobe or toe of the wearer.

In some instances of the method, the one or more processors are not mounted on the flexible electronic tattoo and/or the peripheral sensor.

In some instances of the method, each of the plurality of oxygenation sensors and the peripheral sensor comprise multiple photoplethysmography (PPG) sensors.

In some instances of the method, the plurality of oxygenation sensors, and the peripheral sensor communicate wirelessly with the one or more processors.

In some instances of the method, the flexible electronic tattoo does not apply any pressure to the location on the skin of the wearer.

In some instances of the method, the information related to the in-situ blood oxygenation from within the vein of the wearer and/or the information related to the in-situ blood oxygenation from within the artery of the wearer comprise venous and arterial pulse waveforms, respectively.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIGS. 1A and 1B illustrate examples of an e-tattoo wearable used for in vivo measurements, as described herein.

FIG. 1C shows an overview of an exemplary wearable device on a flexible PCB capturing both arterial and venous waveforms with a PPG sensor array and processing components.

FIG. 2A illustrates arterial and venous waveforms obtained in vivo, where the vertical dashed line denotes the systolic peak.

FIG. 2B illustrates an example of in vivo R values and estimated SxO₂ acquired from the sensor (above artery and vein, respectively). This is without spatial filtering.

FIG. 3 illustrates examples of waveform changes with and without pressure showing differences in arterial and venous waveform shape.

FIG. 4 illustrates examples of estimations of absorption crosstalk between artery and vein using light propagation in tissue simulations.

FIG. 5A illustrates an exemplary phantom setup; and FIG. 5B illustrates an example target and achieved μₐ spectrum for blood to create varying blood oxygen saturations.

FIG. 6 illustrates examples of acquired R values from a multivessel dynamic phantom with true R values from the vein and artery motors independently running and the R values with both motors simultaneously running, showing the effects of crosstalk on R value distributions.

FIG. 7 illustrates an example of simulating the improvement of spatial resolution using spatial filtering.

FIG. 8 illustrates that R values are greatly improved using spatial filtering.

FIGS. 9A-9C illustrate a phantom to mimic human tissue mechanics and optical properties, where FIG. 9A illustrates the setup: 1. Oxygenated blood, 2. Phantom, 3. Vibration dampener, 4. PPG array, 5. Deoxygenated blood, 6. Syringe, 7. Pump; FIG. 9B illustrates am overhead view of PPG array and vessels: i. Phantom, ii. Oxygenated vessel, iii. LEDs, iv. PDs, v. Deoxygenated vessel; and FIG. 9C illustrates vessel dynamics: Expansion during inflow (top), contraction during outflow (bottom).

FIG. 10 illustrates acquired true and mixed R values from the dynamic skin phantom of FIGS. 9A-9C.

FIG. 11 shows acquired mixed and filtered R values using Laplacian filter from the dynamic skin phantom.

FIG. 12 shows acquired mixed and filtered R values using a custom spatial filter from the dynamic skin phantom.

FIG. 13 shows acquired raw mixed and filtered R values using a custom spatial filter from in vivo data at the wrist.

FIG. 14 is an illustration of an exemplary system for pressureless, non-invasive system for simultaneous extraction and measurement of arterial and venous blood oxygenation.

FIG. 15 illustrates an example of an electronic tattoo, which can be used according to embodiments described herein.

FIG. 16 shows an example computing environment in which example embodiments and aspects may be implemented.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

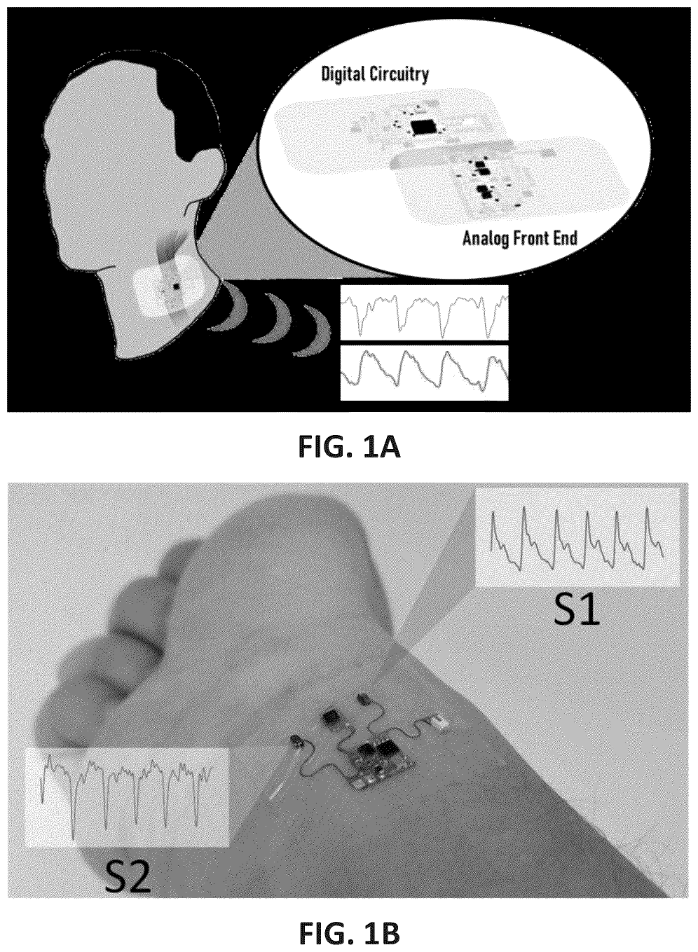

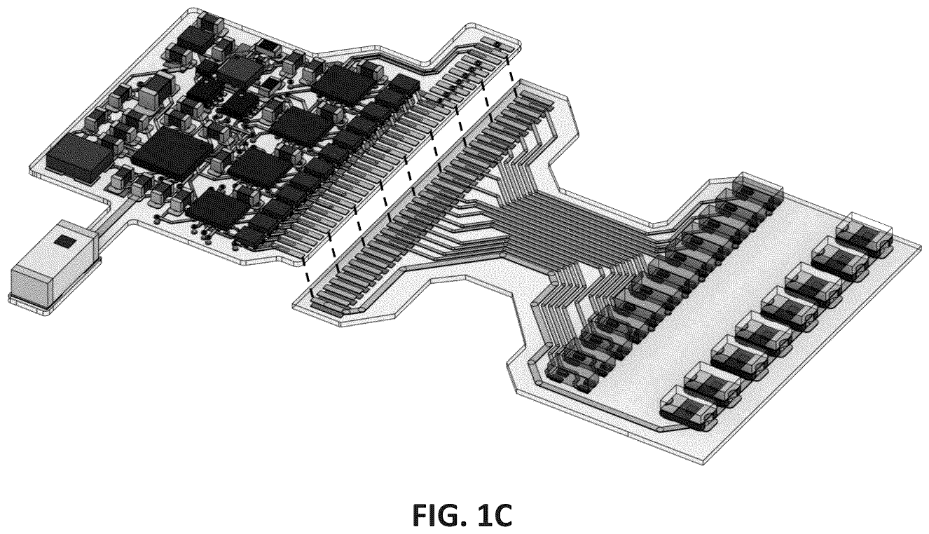

Examples of an e-tattoo wearable used for in vivo measurements are shown in FIGS. 1A and 1B. FIG. 1C shows an overview of an exemplary wearable device on a flexible PCB capturing both arterial and venous waveforms.

The disclosed e-tattoo comprises a plurality of photoplethysmography (PPG) sensors, comprising a source or a plurality of sources configured to transmit multiple wavelengths (for example, λ=535 nm, 660 nm, 940 nm, 950 nm, etc.) that are detected by a plurality photodiode detectors. In some instances, embodiments of the sensor captures PPG signals at two wavelengths (660 nm and 940 nm) using an array design comprising of eight photodiodes (PDs) and 24 LEDs that, which provides 48 channels of data. Each detector's signal is amplified, filtered, and digitized by a custom integrated analog front end and analog-to-digital converter (e.g., MAX86141, Maxim Integrated). The digitized signals are then transmitted over Bluetooth Low Energy (BLE) with a Bluetooth microcontroller (e.g., nRF52811) to a mobile application (for example, a custom Android application). Generally, the system is powered with a standard Lithium Polymer (LiPo) battery, sized for the application. In FIG, 1A, the e-tattoo is shown worn on the neck such that simultaneous jugular vein and carotid artery pulse waveforms are monitored. In FIG. 1B, the e-tattoo is shown worn on the wrist capturing simultaneous arterial and venous waveforms.

In one aspect, the disclosed PPG sensor array, designed for flexible placement over artery/vein pairs, captures data across 48 channels (24 red and 24 IR). The array first identifies two locations with the most significant changes in detected light intensity, indicative of positions directly above the artery and vein. Neighboring channels with intensities within 70% of these peak values are selected for further analysis. Data from these selected channels are processed through a bandpass filter (e.g., 0.5 Hz to 5 Hz) and segmented into 2-second windows.

It is to be appreciated that systems and devices having fewer or greater PPG sensor arrays and data channels are contemplated within the scope of this disclosure.

As described herein, all waveforms are represented as changes in absorbance (ΔA) rather than received light intensity (I) to represent volumetric blood changes. ΔA is calculated through the Modified Beer-Lambert Law (MBLL)

$$Δ A = log ( I ( t ) / I ( t = 0 ) ) . \qquad ( 1 )$$

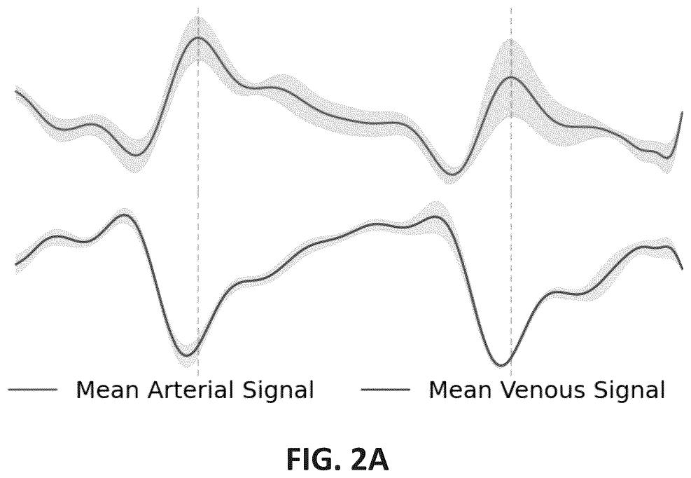

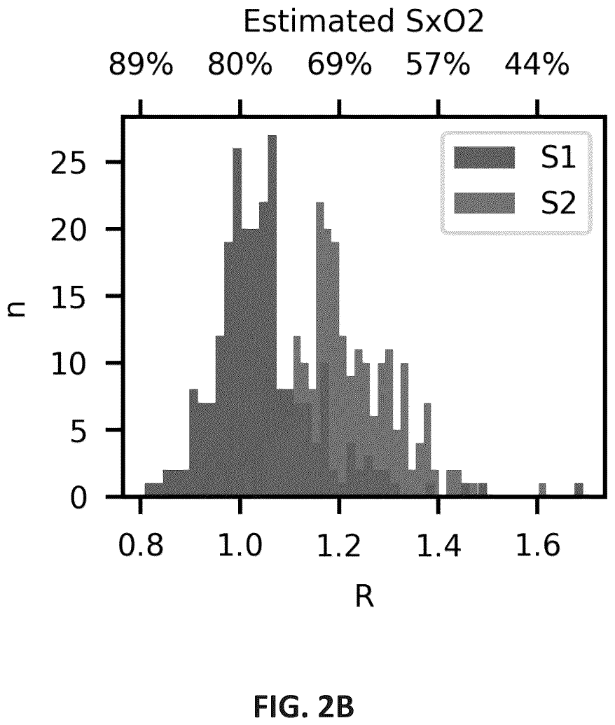

When placed over major (e.g., radial) vessels, it was observed that the PPG waveform morphology is highly position dependent. Specifically, the PPG waveform may appear inverted, with a prominent descent (i.e., decrease in blood volume) rather than the expected upstroke (i.e., increase in blood volume) associated with systole. This is physiologically explained if the PPG sensor is capturing venous blood volume variations. Immediately before systole, the right atrium relaxes and rapid atrial filling leads to a sudden reduction in venous blood volume. In FIG. 1B, two simultaneously obtained waveforms are shown (S1, S2) with apparent morphology differences. Herein, waveforms are referred to as appearing more arterial or venous based on the presence of systolic upstroke or x′ descent, respectively. Referring to FIG. 2A, the arterial waveform is characterized by a systolic peak, representing the maximum pressure during a heartbeat, followed by a diastolic trough, indicating the minimum pressure. In contrast, the venous waveform in healthy individuals is marked by three distinct peaks (a, c, and v) and two descents (x and y). Before the onset of systole, the right atrium relaxes, leading to a sudden reduction in venous blood volume. Consequently, a decrease in venous blood volume precedes the systolic upstroke, as observed in the waveforms of FIG. 2A.

Using red (660 nm) and infrared (940 nm) waveforms from FIG. 1B, SₓO₂ was calculated for the two positions using the ratio-of-ratios (R), where

$$R = Δ {A}_{660 nm} / Δ {A}_{940 nm} . \qquad ( 2 )$$

Where R is commonly approximated as:

$$R \approx ( {AC}_{660 nm} / {DC}_{660 nm} ) / ( {AC}_{940 nm} / {DC}_{940 nm} ) . \qquad ( 3 )$$

Herein, R is calculated in its complete form as written in Equation (2). The R value is related to blood oxygen saturation (SₓO₂) using an empirically derived calibration curve. R decreases monotonically with increasing SxO₂. To give a rough estimate of the corresponding SxO₂, R values are converted using a predefined calibration curve [see, J. P. de Kock and L. Tarassenko, “Pulse oximetry: Theoretical and experimental models,” Medical and Biological Engineering and Computing, vol. 31, no. 3, pp. 291-300, 1993/05/01 1993, which is fully incorporated by reference]. However, note that this calibration curve is accurate only for a specific source-detector distance and sensor placement (i.e., finger). Therefore, the listed SxO. values herein should only be viewed as rough estimates. Significantly different (P<0.001) R distributions obtained from the two measurement positions (FIG. 2B) were observed. However, each distribution has high variability and significant overlap. It is hypothesized that the poor distinguishment between SaO₂ and SvO₂ is largely caused by crosstalk between the two vessels. Since light diffuses throughout a large volume of tissue due to scattering, light from each sensor will interact with both vessels and therefore lead to a saturation representing a mixture of both.

It is hypothesized that PPG waveforms obtained from the wrist are the superposition of arterial and venous pulses. Varying sensor placement alone leads to signals that have more arterial or venous pulse characteristics. However, pulse waveform comparison is relatively subjective and qualitative. Although the waveforms have been previously compared to diameter waveforms obtained through ultrasound, it has not been proven that the vein is the true cause of waveform morphology change.

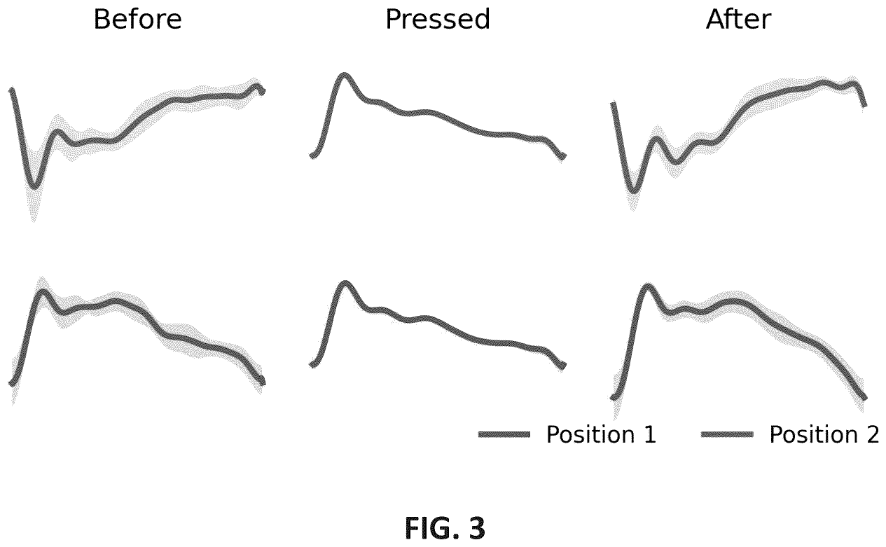

To determine if the source of the signal was from venous pulsatility, PPG signals from the wrist were recorded at two locations with and without applied pressure (see FIG. 3). Signals were segmented at the same time points for every cardiac cycle, normalized in time and amplitude, and are displayed as a grand average as a dark line. The standard deviation is plotted as shaded regions. As the internal pressure in the veins is very low (10-20 mmHg), externally applied pressure should collapse the vein and prevent it from pulsing. Without applied pressure (i.e., before and after applying pressure) to the wrist, the signal from Position 1 has strong venous characteristics while the signal from Position 2 has strong arterial characteristics. With applied pressure to the wrist, the signals from both positions become nearly identical and appear arterial. The beat-to-beat variability of the waveforms (i.e., standard deviation) also greatly decreases. These results suggest that the change in waveform does come from venous pulsatility. Additionally, although waveforms may appear solely arterial or venous, they still contain signal components from both vessels.

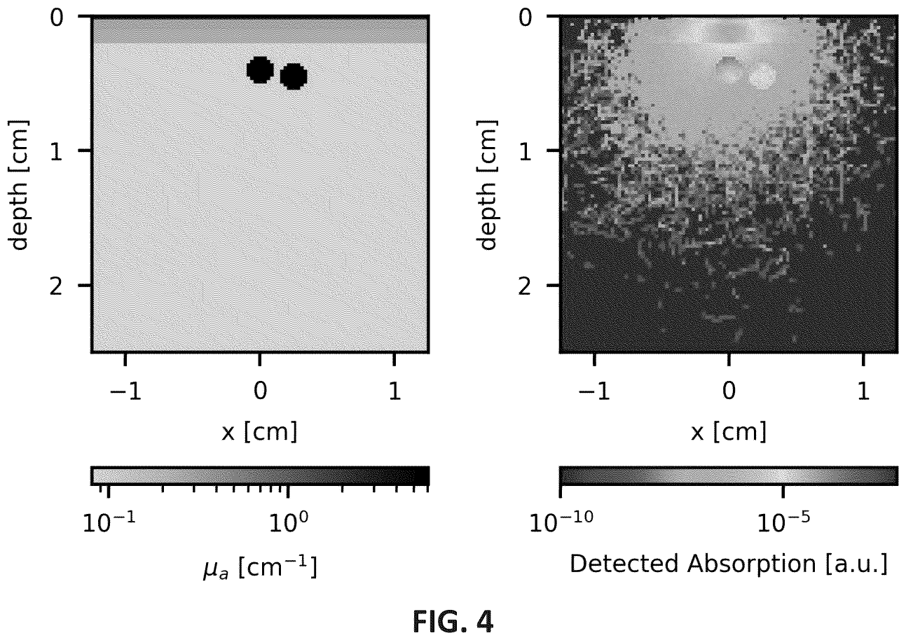

Monte Carlo simulations (MCmatlab) were used to model the photon propagation in the tissue. A region of tissue containing parallel, cylindrical blood vessels was molded. The source-detector distance was set to 6 mm and tissue optical properties for 950 nm light were calculated. In order to characterize the amount of crosstalk the vein will have on the artery, the spatial distribution of absorption for light that reached the detector was calculated. First, the fluence (F) distribution received by the detector was extracted. Multiplying this F distribution voxel-wise by the tissue absorption coefficients yields the absorption in the optical path (FIG. 4) shows an estimation of absorption crosstalk using simulations. The simulated artery and vein contributed 17.9% and 4.4%, respectively, to the total observed absorption. Assuming only the blood vessels induce AC absorption signals, this demonstrates that an off-center secondary vessel can contribute up to 20% of the total observed pulsatile absorption. As a result, despite centering the sensor over a single target vessel, the neighboring vessel will induce significant crosstalk.

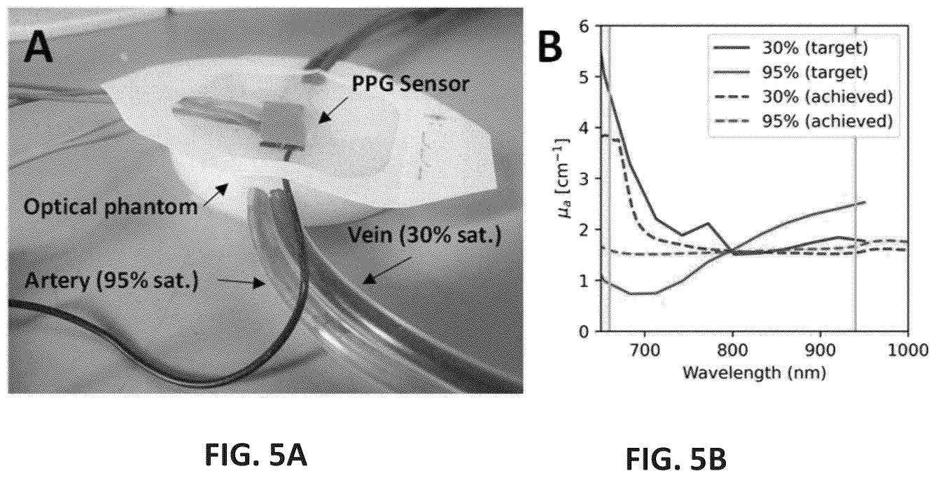

Initial in vivo results suggest that crosstalk between the artery and vein cause the measured SaO₂ and SvO₂ to converge. To test this hypothesis, a dynamic optical phantom (FIG. 5A) was constructed. The phantom was created by doping PDMS with TiO₂ to 0.1% wt. Artificial blood was pumped through the phantom and was created by adding dyes and mica pigments to water. The absorption spectra of the fake blood were set to mimic a higher saturation (95%) and a lower saturation (30%) by tuning the concentrations of the constituent dyes and pigments. Although in vivo SvO₂ is typically around 80%, the targeted in vivo SvO₂ was 30% as there were challenges obtaining an isosbestic point using the current set of chromophores. The chromophore concentrations were calculated through a custom gradient descent optimization algorithm. The absorption coefficients (μa) for the phantom blood were verified using a laboratory UV/VIS spectrophotometer (AccuSkan Go, Fischer Scientific) and are displayed in FIG. 5B. Although an isosbestic point around 800 nm like real blood was achieved, it is thought that the accuracy of the achieved absorption spectra may be improved by using a wider range of dyes and pigments.

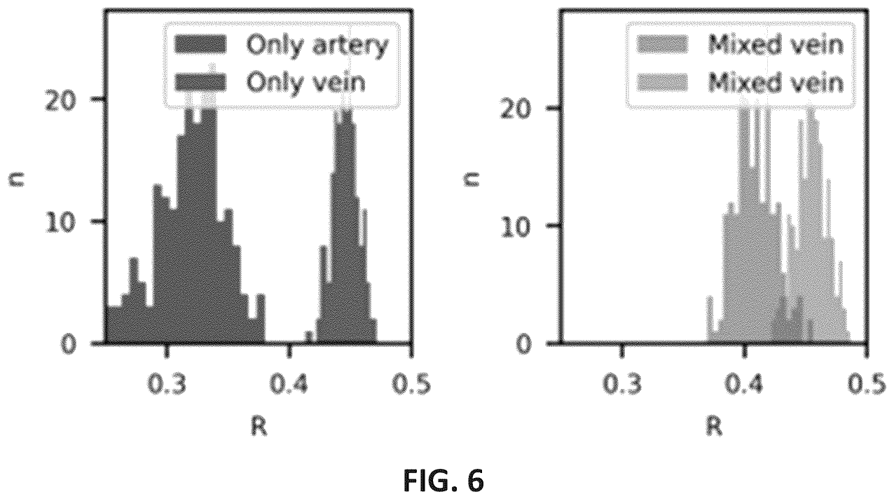

The PPG sensors were placed on the phantom directly over the vessels and measured absorption signals with the artificial blood pumped through just the artery, just the vein, and both vessels simultaneously. Distributions of acquired R values for all three setups are shown in FIG. 6. When measuring the single vessels, the sensor acquired well separated R distributions (labeled “Only artery” and “Only vein”). While measuring both vessels simultaneously, the acquired R distributions (labeled “Mixed artery” and “Mixed vein”) converge, replicating what we observed in our initial in vivo experiments. This supports the hypothesis that crosstalk between vessels causes poor SaO₂ and SvO₂ distinction.

Spatial filtering is a common method for reducing blur in images and improving spatial resolution of electroencephalogram (EEG) signals. Here, spatial filtering is used to reduce crosstalk between the two vessels. Using Monte Carlo simulations, the detected F distribution is calculated for a single source-detector for a region of tissue without vessels. As a first pass, it was assumed that the F pattern is spatially invariant; however, inhomogeneous internal anatomy will lead to spatially variant F patterns. This F distribution was copied and shifted to mimic the F patterns acquired from adjacent source-detectors. The pitch between source-detectors 1.25 mm. The detected absorbances from the adjacent source-detectors were used to spatial filter the center. In one instance, a Laplacian filter may be used, where

$${F}_{i} , filtered = 2 {F}_{i} - {F}_{i} - 1 - {F}_{i} . \qquad ( 4 )$$

to separate the absorbance from the artery and vein. In other instances other filtering techniques can be used.

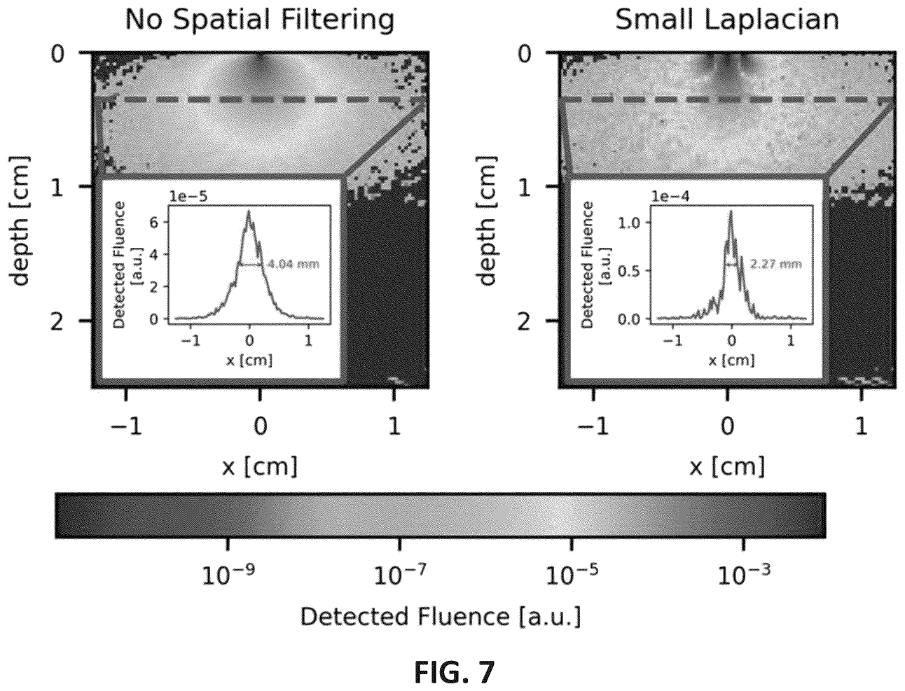

The Full Width at Half Maximum (FWHM) improved from 4.04 mm to 2.27 mm at a depth of 3.5 mm, the approximate depth of the radial vessels. FIG. 7 illustrates the improved spatial resolution of the Laplacian filter.

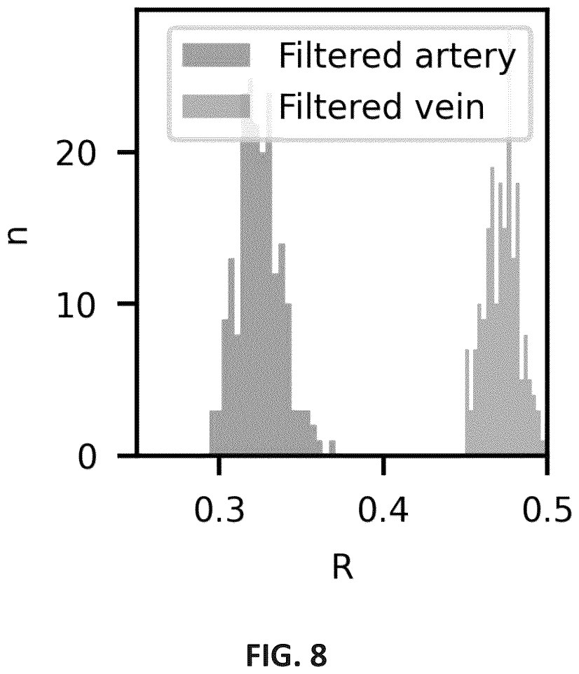

The above-described mixed artery and mixed vein data was spatially filtered. The detected light intensities were converted into absorbances with Equation (1), above, before spatial filtering as the true origin of the crosstalk is blurred absorbances. Therefore, as mentioned herein, Equation (2) was used to calculate R because it is a direct function of absorbances, not light intensities. The obtained R values for the artery and vein after spatial filtering (labeled “Filtered artery” and “Filtered vein”, respectively) are shown in FIG. 8, and were able to be corrected back to the single vessel measurements obtained above.

Building on the above testing of an exemplary e-tattoo, which was described in [Philip Tan, Eric Wang, Shreya Tamma, Sarnab Bhattacharya, and Nanshu Lu. Towards simultaneous noninvasive arterial and venous oxygenation monitoring with wearable e-tattoo. In 2023 45th Annual International Conference of the IEEE Engineering in Medicine & Biology Society (EMBC), pages 1-4. IEEE, 2023, which is fully incorporated by reference and made a part hereof], it is shown below that that an expanded PD-LED array provides enhanced spatial resolution, which improves the efficacy of spatial filtering techniques and yields a better reduction of crosstalk effects.

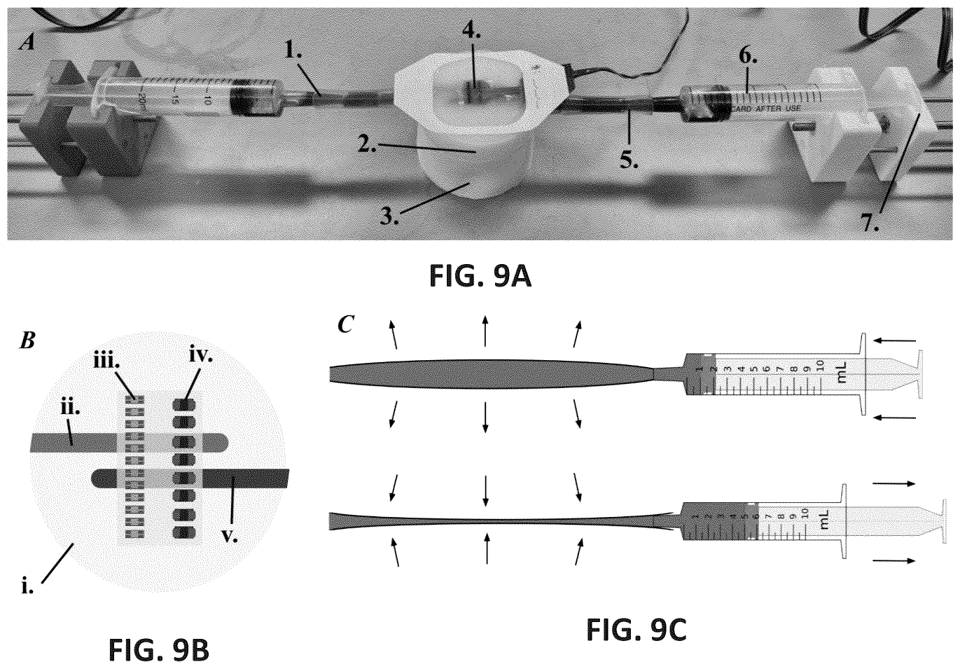

To mimic human tissue mechanics and optical properties, a dynamic skin phantom (as shown in FIGS. 9A-9C) was developed using polydimethylsiloxane (PDMS) with a base-to-curing agent ratio of 20:1, achieving a Young's modulus of 0.5 MPa to closely resemble human tissue. The phantom was doped with 0.1% wt. titanium dioxide (TiO₂) to simulate the optical properties of human skin. It includes two parallel cylindrical channels, each 5 mm in diameter and 2.5 mm beneath the surface, designed to simulate an artery/vein pair. The pumping action induces the expansion and contraction of the simulated vessels, replicating the physiological pulsatile behavior of real blood vessels. Synthetic blood, formulated by mixing water, dyes, and mica pigments to replicate the optical spectra of oxygenated and deoxygenated blood, is pumped through these channels. The dye and pigment concentrations were optimized using the GEKKO Python library. A custom-built syringe pump system, driven by stepper motors and controlled through an Arduino Nano, generates pulsatile flow, mimicking vascular dynamics.

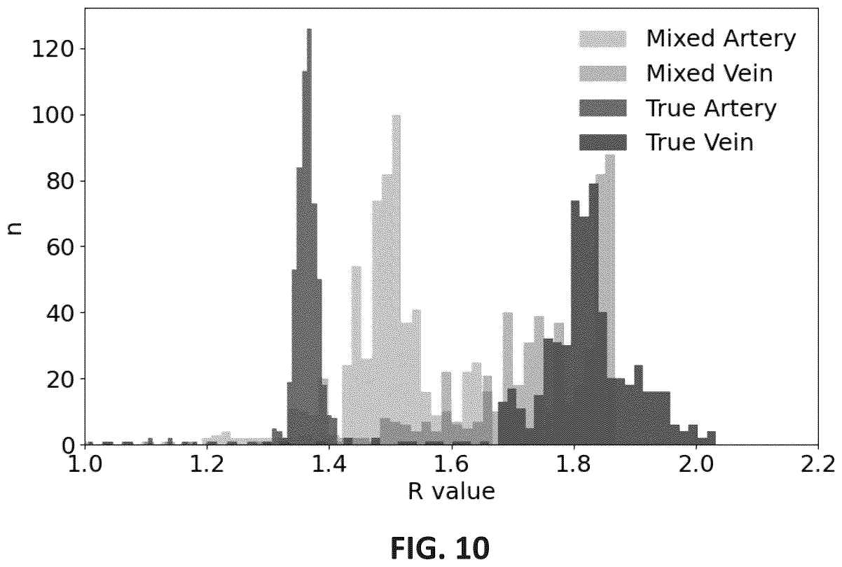

The sample PPG sensor array was placed over two simulated vessels within the phantom, capturing light-intensity signals with synthetic blood pumped independently into each vessel and then both simultaneously. The resulting R value distributions for all three scenarios are illustrated (FIG. 10). When only one vessel was active, two distinct R value distributions were observed, corresponding to each vessel. However, with both vessels simultaneously active, a noticeable shift was detected in both vessel distributions. This indicates that crosstalk may affect the precision of extracting SₐO₂ and SᵥO₂.

A Laplacian filter to enhance signal quality by attenuating the influence of neighboring signals. The filter is defined by the following convolution operation:

$$L ( i ) = {\sum }_{j = 1}^{k} {w}_{j} ( f ( i + j ) + ( f ( i + j ) ) + {w}_{0} f ( i ) \qquad ( 4 )$$

where f(i) denotes the signal value at position i, wⱼ are the weights assigned to the j-th neighbors, decreasing with distance from the center, and wₒ is the central weight, intensified to emphasize the central signal component. The optimal extent of neighborhood inclusion, k=5, was determined by minimizing the Kullback-Leibler (KL) divergence between the true and filtered signal distributions.

While Laplacian filtering can address crosstalk and enhance signal quality, its conventional approach assigns weights without considering the physiological properties of tissue and light scatter. To improve this, we propose a custom spatial filtering algorithm. This method adapts the traditional Laplacian filter by incorporating weights derived from an exponential decay function, aligning more closely with the physiological decay of signal intensity due to tissue scattering.

The adaptive filter is defined as:

$${F}_{i} = {cF}_{i} - a {\sum }_{k = 1}^{N} {w}_{k} ( {F}_{i - k} + {F}_{i + k} ) , \qquad ( 5 )$$

where Fᵢ denotes the signal at the i-th position, c is a scaling factor for the central signal, N represents the number of neighboring points considered, and wₖ are the weights assigned according to the exponential decay wₖ=e^{-λk}, with λ being the decay rate. This formulation ensures that the filter's influence diminishes with distance, providing a response that better mirrors the physical processes of light interaction with biological tissues.

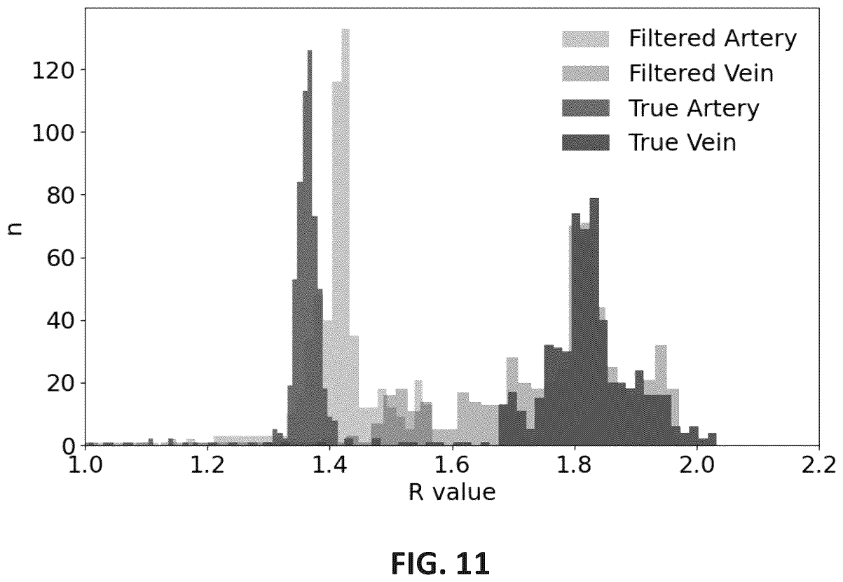

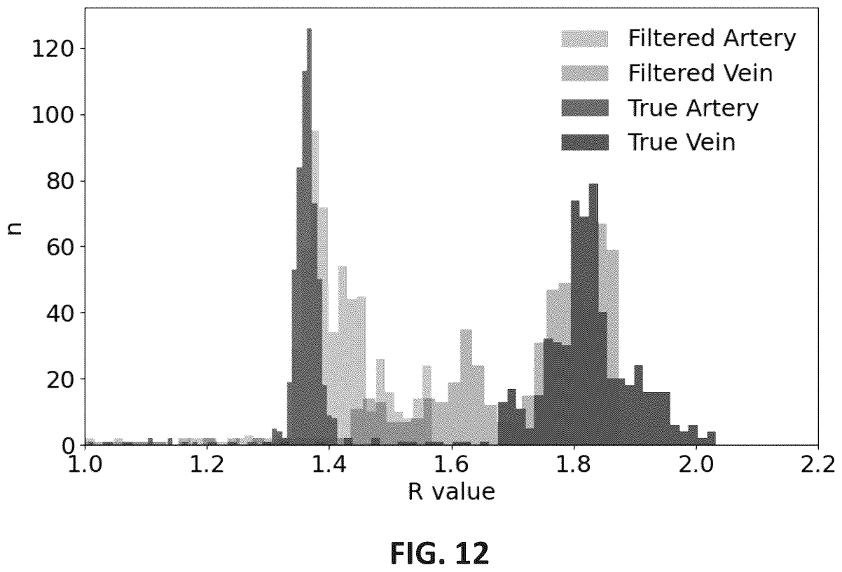

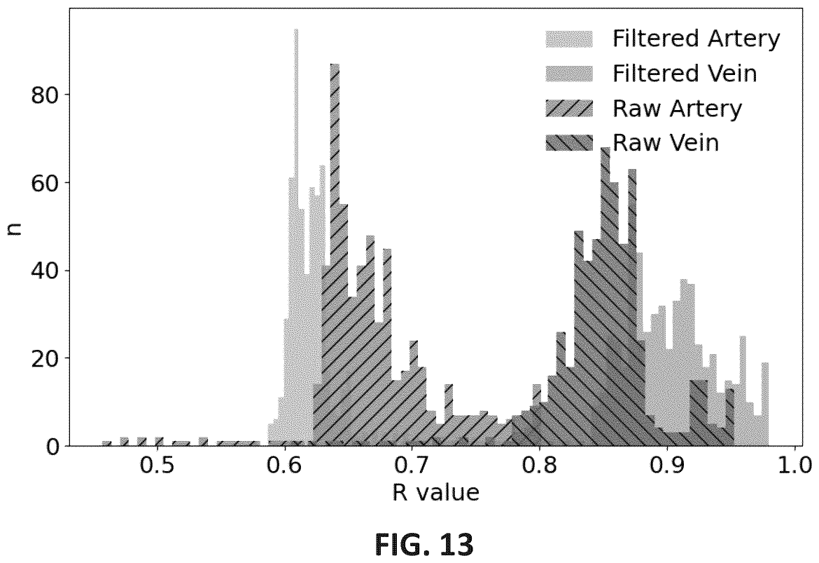

Results of testing using the described phantom as shown in FIGS. 11, 12, and 13, where FIG. 11 shows acquired mixed and filtered R values using Laplacian filter from the dynamic skin phantom; FIG. 12 shows acquired mixed and filtered R values using a custom spatial filter from the dynamic skin phantom; and FIG. 13 shows acquired raw mixed and filtered R values using a custom spatial filter from in vivo data at the wrist.

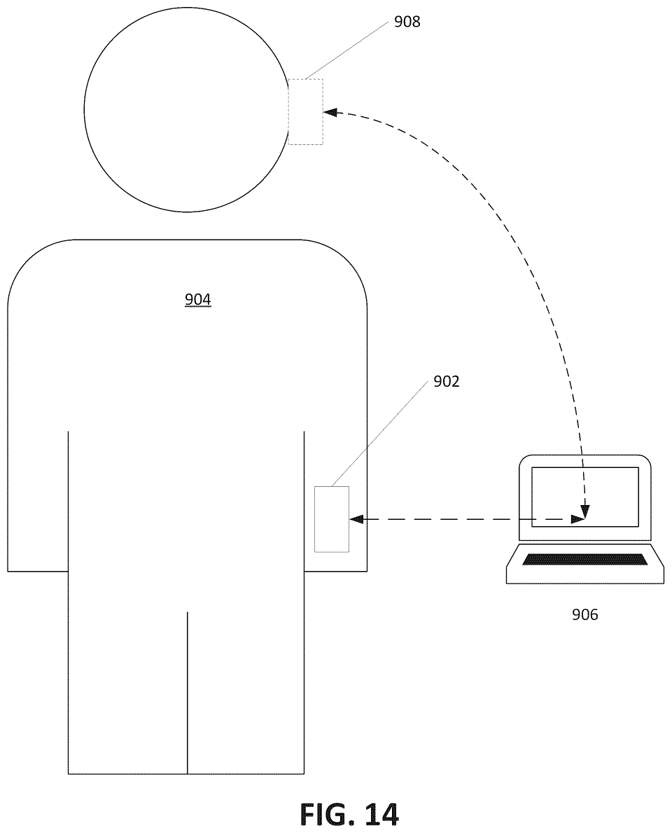

FIG. 14 is an illustration of an exemplary system for pressureless, non-invasive system for simultaneous extraction and measurement of arterial and venous blood oxygenation. The exemplary system comprises a flexible electronic tattoo 902. The flexible electronic tattoo comprises a flexible conformable substrate that is configured to conform to and adhere to a location on a skin of a wearer 904. Further comprising the electronic tattoo are a plurality of photoplethysmography (PPG) sensors that are used to non-invasively obtains information related to in-situ blood oxygenation from within a vein and/or artery of the wearer. The electronic tattoo 902 further comprises a communications interface that is used to communicate with one or more processors 906. Examples of processors include mobile devices, computers, or other body-worn microprocessors. The one or more processors receive the information related to the in-situ blood oxygenation from within the vein of the wearer and/or the information related to the in-situ blood oxygenation from within the artery of the wearer and simultaneously extract venous blood oxygenation (SvO₂) and arterial blood oxygenation (SaO₂) from the information related to the in-situ blood oxygenation from within the vein of the wearer and/or the information related to the in-situ blood oxygenation from within the artery of the wearer.

The one or more processors 906 make a determination about oxygen saturation of a localized area proximate the location on the skin of the wearer 904 where the electronic tattoo 902 is adhered based on the observed optical absorptions. For example, the determination about oxygen saturation may comprise a one-dimensional (1D) distribution of absorbance changes, or a two-dimensional (2D) image of absorbance changes of the localized area. Non-limiting examples of the localized area of the wearer 904 include the neck, chest, back, stomach, upper arm, upper leg, lower leg, and the like of the wearer 904.

Furthermore, in addition to just locating the artery and vein, the plurality of PPG sensors and the corresponding calculation of distribution of absorption changes are used to provide spatial filtering. The information from each sensor is used to modify the estimated oxygenation from nearby sensors (i.e., spatial filtering), which is required because the light diffuses/scatters through a large volume of tissue and the artery and vein are very close to each other. Therefore, there will be crosstalk or a mixture of signals.

In some instances, the system may optionally include a peripheral sensor 908, separate from the flexible electronic tattoo 902, wherein the peripheral sensor 908 non-invasively obtains oxygenation information related to a peripheral site of the wearer and provides it to the one or more processors 906. The one or more processors 906 calibrate the venous oxygenation sensor and/or the arterial oxygenation sensor based on the oxygenation information about the peripheral site. The peripheral site may comprise a fingertip, earlobe, toe, or the like of the wearer 904. In some instances, the peripheral sensor 908 may comprise a PPG sensor.

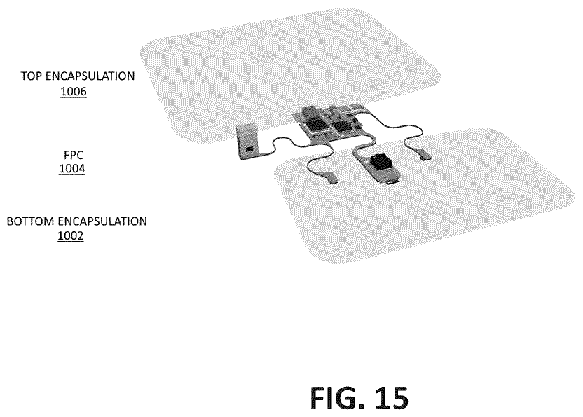

FIG. 15 illustrates an example of an electronic tattoo 902, which can be used according to embodiments described herein. This embodiment comprises a thin, macro-conformal, stretchable, and lightweight sensing platform that can be laminated onto the human skin like a temporary tattoo and hence termed an electronic-or e-tattoo. Wireless connectivity incorporating Bluetooth Low Energy (BLE) is utilized to stream the data in real-time to a host device. As noted above, the disclosed e-tattoo comprises a plurality of optical (photoplethysmogram (PPG)) sensors. In some instances, the e-tattoo has a stacked-design, which facilitates conformability, stretchability and easy fabrication/reuse.

Generally, as shown in FIG. 15, embodiments of the device comprise a flexible printed circuit (FPC) layer 1004 and a cover layer 1006. An additional sacrificial layer 1002 may be included to provide extra adhesion to the skin.

In some instances, the transparent first flexible, stretchable insulating substrate comprises a polyurethane film medical dressing, such as Tegaderm™ (3M, Saint Paul, MN), having an adhesive layer on the first side of the transparent first flexible, stretchable insulating substrate. This layer may be disposed between uses to reuse the electronics on the FPC layer.

Further comprising embodiments of the device is the flexible printed circuit (FPC) layer 1004. In some instances, the FPC layer 104 may be covered with a third flexible substrate 1006 that covers the first flexible, stretchable insulating substrate and the second flexible substrate. In some instances, this third layer of material 1006 may comprise Tegaderm™. In some instances, a portion of the third flexible substrate 1006 may be removed to define one or more holes that expose a power source (e.g., a battery) mounted on the FPC layer 1004. In this way, the power source can be replaced as needed without having to replace the entire device.

Generally, the FPC layer 1004 comprises electronics disposed at least partially on a second side of the second flexible insulating substrate. In some instances, the second flexible insulating layer comprises polyimide. Typically, the first side of the second flexible insulating substrate is in substantial contact with the second side of the first flexible, stretchable insulating substrate. Generally, the electronics comprise at least a wireless communications interface, a central processing unit, and a power source. Components of the electronics are separated from one another on the second side of the second flexible insulating substrate and connected to one another using conductors configured to flex and stretch with the transparent first flexible, stretchable insulating substrate and/or the second flexible insulating substrate, and to conform to the epidermis. For example, the conductors may comprise copper gold traces having a serpentine pattern.

The electronics may further comprise the plurality of photoplethysmogram (PPG) sensors generally disposed on the FPC layer. Generally, each PPG sensor comprises one or more light emitting diodes (LEDs) and one or more photodetectors (PD) that perform reflective photoplethysmography through the transparent first flexible, stretchable insulating substrate. The LEDs and PDs are electrically connected to the electronics disposed at least partially on the second side of the second flexible insulating substrate through the second flexible insulating substrate.

Together, the electronics and the second flexible insulating substrate comprise the FPC layer 1004. It should be noted that the electronics are generally completely isolated from contact with the human body (i.e., epidermis) and any components in direct skin contact are comprised of biocompatible materials.

### Computing Environment

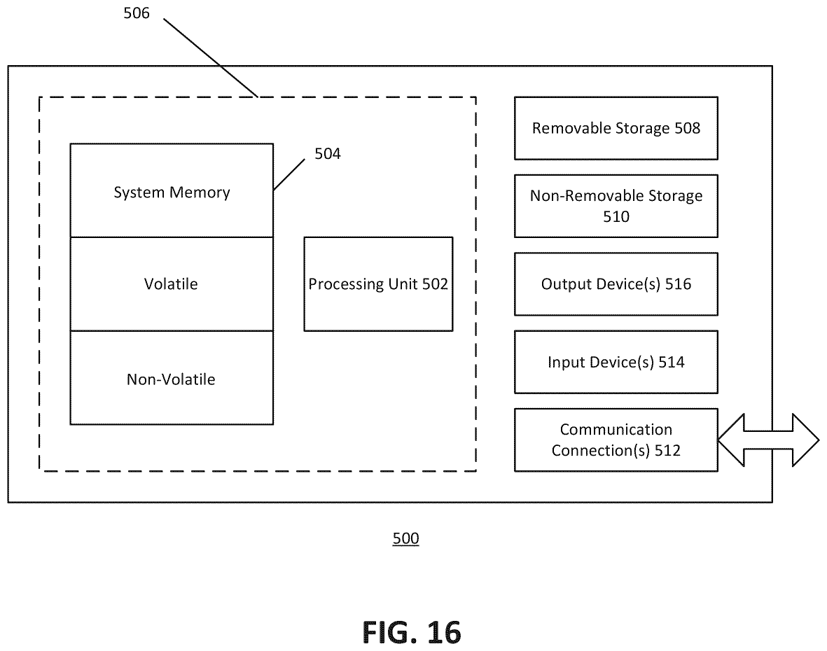

FIG. 16 shows an example computing environment in which example embodiments and aspects may be implemented. The computing device environment is only one example of a suitable computing environment and is not intended to suggest any limitation as to the scope of use or functionality. The computing environment of FIG. 16 may be a computing device 500 used by a controller, and/or other hardware aspects of the disclosure, to implement aspects of the disclosure. For example, computing device 500 may be a component of or comprise a cloud computing and storage system. Computing device 500 may comprise all or a portion of a server or a controller. Each computing device may have one or more processors. In various implementations, computing devices 500 used by the various parties may be interconnected with one another through various connections, including networks. Such networks may be wired (including fiber optic), wireless, or combinations thereof including parallel, RS-232 (all serial communication from point to point), Visual (Infra-Red), audible (modem for example). Other connections/communication standards can also be improved such as USB, PCI Express, Firewire, Fiber Channel, HDMI, I2C, SPI, etc. Some of these are important for applications such as wireless barcode readers, Credit Card Terminals, walkie-talkies, radios, video conferencing, etc.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, cloud-based systems, microprocessor-based systems, network personal computers (PCS), minicomputers, mainframe computers, embedded systems, Internet of Things devices, network switches, network routers, network edge devices, Modulator-demodulators (modems), industrial control equipment, including distributed computing environments that include any of the above systems or devices, smart phones or smart devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 16, an example system for implementing aspects described herein includes a computing device, such as computing device 500. In its most basic configuration, computing device 500 typically includes one or more processing units 502 and one or more memory 504. Depending on the exact configuration and type of computing device, memory 504 may be volatile (such as random-access memory (RAM)), non-volatile (such as read-only memory (ROM), flash memory, etc.), or some combination of the two. This most basic configuration is illustrated in FIG. 16 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 16 by removable storage 508 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by computing device 500. Any such computer storage media may be part of computing device 500.

Computing device 500 may contain communication connection(s) 512 that allow the device to communicate with other devices over networks. Such networks may be public or private, combinations thereof, and may include the internet. Computing device 500 may also have input device(s) 514 such as a keyboard, mouse, pen, voice input device, touch input device, etc. Output device(s) 516 such as a display, speakers, printer, etc. may also be included.

It should be understood that the various techniques described herein may be implemented in connection with hardware components or software components or, where appropriate, with a combination of both. Illustrative types of hardware components that can be used include Field-programmable Gate Arrays (FPGAS), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In the specification and/or figures, typical embodiments have been disclosed. The present disclosure is not limited to such exemplary embodiments. Those skilled in the art will also appreciate that various adaptations and modifications of the preferred and alternative embodiments described above can be configured without departing from the scope and spirit of the disclosure.

The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A pressureless, non-invasive system for simultaneous extraction and measurement of arterial and venous blood oxygenation, comprising:
a flexible electronic tattoo, said flexible electronic tattoo comprising:
a flexible conformable substrate, wherein the substrate is configured to conform to and adhere to a location on a skin of a wearer;
a plurality of optical oxygenation sensors mounted on the substrate, wherein the optical oxygenation sensors each non-invasively obtain information related to in-situ blood oxygenation from within a vein and/or artery of the wearer;
a communications interface mounted on the interface, wherein the communications interface is in communication with the plurality of optical oxygenation sensors; and
a power source mounted on the flexible conformable substrate; and
one or more processors, wherein the one or more processors receive the information related to the in-situ blood oxygenation from within the vein and/or artery of the wearer through the communications interface and simultaneously extract venous blood oxygenation (SvO₂) and arterial blood oxygenation (SaO₂) from the information related to the in-situ blood oxygenation from within the vein and/or artery of the wearer.

2. The system of claim 1, wherein the one or more processors make a determination about oxygen saturation of a localized area proximate the location on the skin of the wearer where the electronic tattoo is adhered based on observed optical absorptions.

3. The system of claim 2, wherein the determination about oxygen saturation comprises a one-dimensional (1D) distribution of absorbance changes, or a two-dimensional (2D) image of absorbance changes of the localized area.

4. The system of claim 2, wherein the information related to the in-situ blood oxygenation from within the vein and/or artery of the wearer from each of the plurality of optical oxygenation sensors is used to modify extracted venous blood oxygenation (SvO₂) and arterial blood oxygenation (SaO₂) estimations from nearby sensors (spatial filtering and source separation). This is required because the light diffuses/scatters through a large volume of tissue and the artery and vein are very close to each other, resulting in crosstalk or a mixture of signals.

5. The system of claim 2, wherein the localized area comprises the neck, chest, back, stomach, upper arm, upper leg, or lower leg of the wearer.

6. The system of claim 1, further comprising a peripheral sensor, separate from the flexible electronic tattoo, wherein the peripheral sensor non-invasively obtains oxygenation information related to a peripheral site of the wearer and provides it to the one or more processors, wherein the one or more processors calibrate one or more of the plurality of oxygenation sensors based on the oxygenation information about the peripheral site.

7. The system of claim 6, wherein the peripheral site comprises a fingertip, earlobe or toe of the wearer.

8. The system of claim 6, wherein the one or more processors are not mounted on the flexible electronic tattoo and/or the peripheral sensor.

9. The system of claim 6, wherein each of the plurality of oxygenation sensors and the peripheral sensor comprise multiple photoplethysmography (PPG) sensors.

10. The system of claim 6, wherein the plurality of oxygenation sensors, and the peripheral sensor communicate wirelessly with the one or more processors.

11. The system of claim 1, wherein the flexible electronic tattoo does not apply any pressure to the location on the skin of the wearer.

12. The system of claim 1, wherein the information related to the in-situ blood oxygenation from within the vein of the wearer and/or the information related to the in-situ blood oxygenation from within the artery of the wearer comprise venous and arterial pulse waveforms, respectively.

13. A method for using a pressureless, non-invasive system for simultaneous extraction and measurement of arterial and venous blood oxygenation, the method comprising:
attaching flexible electronic tattoo to an epidermis of a wearer, proximate an artery and a vein of the wearer, said flexible electronic tattoo comprising:
a flexible conformable substrate, wherein the substrate is configured to conform to and adhere to a location on a skin of a wearer;
a plurality of optical oxygenation sensors mounted on the substrate, wherein the optical oxygenation sensors each non-invasively obtain information related to in-situ blood oxygenation from within a vein and/or artery of the wearer;
a communications interface mounted on the interface, wherein the communications interface is in communication with the plurality of optical oxygenation sensors; and
a power source mounted on the flexible conformable substrate; and
receiving, by one or more processors, the information related to the in-situ blood oxygenation from within the vein and/or artery of the wearer through the communications interface; and
simultaneously extracting, by the one or more processors, venous blood oxygenation (SvO₂) and arterial blood oxygenation (SaO₂) from the information related to the in-situ blood oxygenation from within the vein and/or artery of the wearer.

14. The method system of claim 12, wherein the one or more processors make a determination about oxygen saturation of a localized area proximate the location on the skin of the wearer where the electronic tattoo is adhered based on observed optical absorptions.

15. The method of claim 13, wherein the determination about oxygen saturation comprises a one-dimensional (1D) distribution of absorbance changes, or a two-dimensional (2D) image of absorbance changes of the localized area.

16. The method of claim 13, wherein the information related to the in-situ blood oxygenation from within the vein and/or artery of the wearer from each of the plurality of optical oxygenation sensors is used to modify extracted venous blood oxygenation (SvO₂) and arterial blood oxygenation (SaO₂) from nearby sensors (spatial filtering and source separation). This is required because the light diffuses/scatters through a large volume of tissue and the artery and vein are very close to each other, resulting in crosstalk or a mixture of signals.

17. The method of claim 13, wherein the localized area comprises the neck, chest, back, stomach, upper arm, upper leg, or lower leg of the wearer.

18. The method of claim 12, further comprising a peripheral sensor, separate from the flexible electronic tattoo, wherein the peripheral sensor non-invasively obtains oxygenation information related to a peripheral site of the wearer and provides it to the one or more processors, wherein the one or more processors calibrate one or more of the plurality of oxygenation sensors based on the oxygenation information about the peripheral site.

19. The method of claim 17, wherein the peripheral site comprises a fingertip, earlobe or toe of the wearer.

20. The method of claim 17, wherein the one or more processors are not mounted on the flexible electronic tattoo and/or the peripheral sensor.

21. The method of claim 17, wherein each of the plurality of oxygenation sensors and the peripheral sensor comprise multiple photoplethysmography (PPG) sensors.

22. The method of claim 17, wherein the plurality of oxygenation sensors, and the peripheral sensor communicate wirelessly with the one or more processors.

23. The method of claim 12, wherein the flexible electronic tattoo does not apply any pressure to the location on the skin of the wearer.

24. The method of claim 12, wherein the information related to the in-situ blood oxygenation from within the vein of the wearer and/or the information related to the in-situ blood oxygenation from within the artery of the wearer comprise venous and arterial pulse waveforms, respectively.
